(19) United States
(12) Patent Application Publication
(10) Pub. No.: US 2008/0027507 A1
(43) Pub. Date: Jan. 31, 2008

(54) Nerve And/Or Muscle Stimulation Electrodes

(75) Inventors: Goran Bijelic, Belgrade (YU); Morten Haugland, Aaiborg (DK)

Correspondence Address: PILLSBURY WINTHROP SHAW PITTMAN, LLP, P.O. BOX 10500, MCLEAN, VA, 22102, US

(73) Assignee: Neurodan A/S, Alborg (DK)

(21) Appl. No.: 10/587,524

(22) PCT Filed: Feb. 1, 2005
(86) PCT No.: PCT/EP05/00943

§ 371 Date: May 30, 2007

(30) Foreign Application Priority Data
Feb. 5, 2004 (GB) 0402569.8

(51) Int. Cl. A61N 1/32

(52) U.S. Cl. 607/48

(57) ABSTRACT

A nerve or muscle stimulation apparatus including a nerve stimulation array electrode including a substrate for application to the skin of a user bearing an array of electrodes arranged to be brought into electrical contact with the skin of a user when the substrate is positioned on the skin; one or more input contacts, the or each input contact being switchably electrically connected to a group of electrodes including some or all of the electrodes; and a user operable switch for performing the electrical connection between the or each input contact and any selected one or ones of its respective group of the electrodes.

The present invention relates to the biomedical arts, particularly neuromuscular electrical stimulation (NMES) by means of surface electrodes and includes apparatus for interfacing an electronic stimulator and a surface electrode with multiple contacts. The invention facilitates the selection of the most appropriate positions of electrodes used for stimulation of sensory-motor systems in humans.

Electrical stimulation of nerves and muscles by means of surface electrodes is now being used for various applications, including pain reduction, muscle strengthening, activation of paralysed muscles, spinal cord stimulation and training of sensory-motor mechanisms. Common to all uses of surface electrodes for stimulation is that it requires a great deal of skill and patience of the user and/or the therapist to place the electrodes in the optimal position for the function to be performed. It is impossible to know precisely the pathways of the electrical charge that should be delivered to sensory-motor systems under the skin. It is therefore difficult to predict precisely which anatomic structures will be activated for any given position and electrode configuration. Very often self-adhering electrodes are used, which must be taken off completely before they can be repositioned at a different location on the skin. This process is not only frustrating and time-consuming, but can also be painful and compromises the adhesion of the electrode to the skin, leading to an increased consumption of electrodes. Further, it is difficult to try many different electrode sizes, even if this may have an important effect on the function and subjective perception of the stimulation. For these reasons a non-optimal electrode position and electrode size are often chosen.

The present invention addresses this problem of the effective use of surface electrodes. It is proposed to use a multiple contact surface stimulation electrode combined with an easy-to-use interface, which allows the user to rapidly emulate many different electrode sizes and positions, without actually removing the electrode from the skin during the process.

U.S. Pat. No. 6,564,079 discloses an electrode array comprising a common adhesive substrate carrying a number of spaced electrodes for use in mapping nerve locations. The electrode array is to be connected to a peripheral nerve detection instrument, the nature of which is not described. Contacts lead from each electrode to electronic circuitry comprising multiplexers and a shift register, but again the exact purpose of this circuitry is not described. It is not disclosed that nerves or muscles should be stimulated or that any voltage is to be applied to the electrodes.

The present invention now provides a nerve or muscle stimulation apparatus comprising:
a nerve stimulation array electrode comprising a substrate for application to the skin of a user bearing an array of electrodes arranged to be brought into electrical contact with the skin of a user when the substrate is positioned on said skin,
one or more input contacts, the or each said input contact being switchably electrically connected to a group of electrodes consisting of some or all of said electrodes, and
user operable switch means for performing said electrical connection by making and breaking electrical connection between the or each said input contact and any selected one or ones of its respective group of said electrodes.

Thus, the array of electrodes provided by the invention allows the array to be positioned generally in an area in which precise stimulation of underlying nerves or muscles is required and for the array to be configured in situ by the selection of an appropriate electrode, or appropriate electrodes as a group or a number of groups, which are ideally placed to achieve the desired effects. There is therefore no need to establish the precise position at which electrical stimulation is required before the electrode is placed or to remove and replace the electrode to achieve the desired position. Instead, the array may be placed in approximately the required position and then configured as described above. One therefore avoids the need for extensive trial and error positioning of electrodes. One is therefore able to select one or more ideally placed contacts as an interface for the delivery of controlled amounts of electrical charge to sensory-motor systems.

The electrodes may be arranged on the substrate in a random manner or in any desired pattern, but it will generally be convenient if said substrate bears an array of electrodes arranged in rows and/or columns. This includes arrangements in which not every point in a row and column array is filled, e.g. hexagonal arrays.

Preferably, there are at least 12 electrodes in the array, more suitably at least 18 or at least 24 electrodes in the array. However, there may be fewer electrodes than this, e.g. 6 or more. The fewer electrodes there are, the more accurate will need to be the placement of the array.

Normally it will be preferable that the electrodes are evenly spread over the substrate. This will maximise the chance that one or several of the electrodes are in the ideal position.

Preferably, the maximum space between adjacent electrodes is no more than 5 cm. More preferably, it is less, e.g. no more than 2 cm. However, the spacing is preferably substantially less, e.g. no more than 1 cm. Still more preferably, the spacing does not exceed 3 mm. Suitably, the spacing is about 1 mm or does not exceed 1 mm.

Preferably, each electrode is not more that 5 cm in maximum dimension, more preferably no more than 2 cm, e.g. about 1 cm.

The size and spacing of the electrodes is suitably chosen to ensure that the charge delivered and the current density will be within safety margins and standards taking into account the stimulation pattern. Relevant parameters to take into account will include the pulse duration and pulse amplitude.

The switch means may be carried by the substrate. Alternatively, the switch means may be carried by a separate controller. Suitably, the switch means comprises a respective manually operable switch associated with each electrode.

Optionally, the switch means comprises:
an electronic user interface unit adapted to receive a user input representing an electrode selection and to communicate data signals representing said selection to an electronic switching unit; and
a said electronic switching unit adapted to receive said data signals and to perform connection of selected electrodes to said input contact(s).

Said electronic user interface unit may be adapted to accept programming to alter said selection of electrodes to accommodate said selection to alterations in the desired site of stimulation with time or user muscle movement. The desired surface position of stimulation may alter as the patient's limb moves or as the relevant muscles move under the skin surface upon stimulation. It should be appreciated also that one of the problems experienced in this field is that repeated use of nerve or muscle stimulation apparatus is that by exercising muscles or muscle groups the geometry of the relevant body part is changed, so that previously ideal electrode sites become less than ideal. The array electrode of the invention allows for these changes to be accommodated either by responding to the changes with reconfiguration of the electrode array in a trial and error manner from time to time or by a pre-programmed reconfiguration.

Optionally, said electronic switching unit is mounted to said substrate and said electronic user interface unit is remote from said substrate and is wirelessly connected or in wired connection with said electronic switching unit.

In addition to said array electrode, the apparatus may comprise one or more further electrodes connected to said switch means. In particular, there may be a common ground or counter electrode, which will not generally need also to be in the form of an array, but may be if desired.

Preferably, the user operable switch means is operable to make connection between the or a said input contact and a first electrode set constituted by a selected one or selected ones of its respective group of electrodes and with a second electrode set constituted by a selected one or selected ones of its respective group of electrodes, leaving disconnected at least one further electrode positioned between said first and second electrode sets. For instance, it has been noticed that the pattern chosen for stimulation of finger extensors is not a simple geometric shape. It may consist of two separate groups of active electrodes, such that one activates two separate muscles that both stretch the fingers whilst not activating the muscle in between, which extends the wrist. For situations like this, control arrangements that allow discontinuously arranged electrodes to be grouped for simultaneous activation, such as individual on/off switches for each electrode, may be preferred over arrangements that only allow adjacent electrodes to be grouped.

The apparatus may further comprise a source of nerve or muscle stimulation signals connected to the or a said input contact.

The invention will be further described and illustrated with reference being made to the accompanying drawings in which:

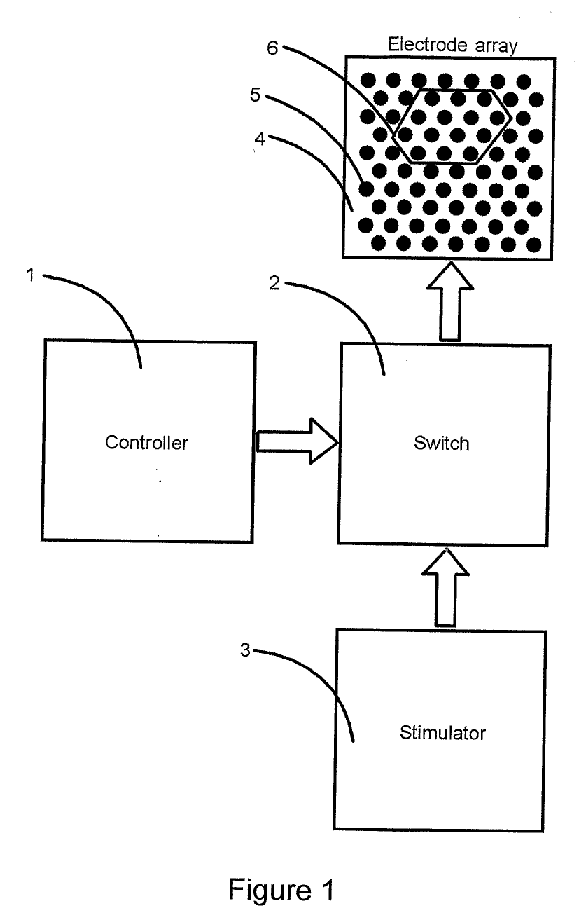

FIG. 1 is an organisational schematic of apparatus for use in the invention;

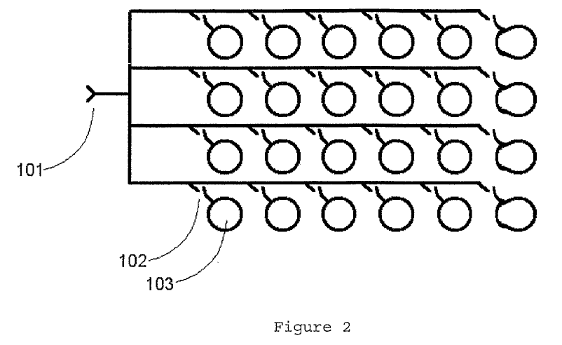

FIG. 2 is a schematic electrical circuit of an array electrode incorporating switches for use in a first embodiment of the invention;

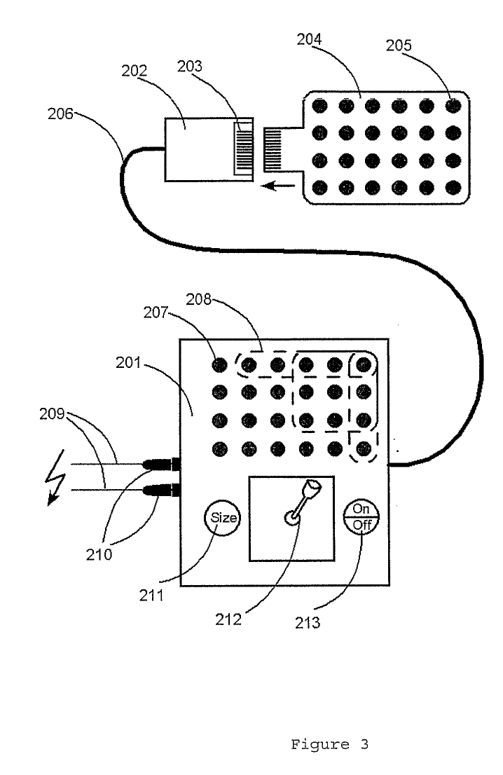

FIG. 3 illustrates a second embodiment of the invention and shows an array electrode in plan view and a control unit also in plan view;

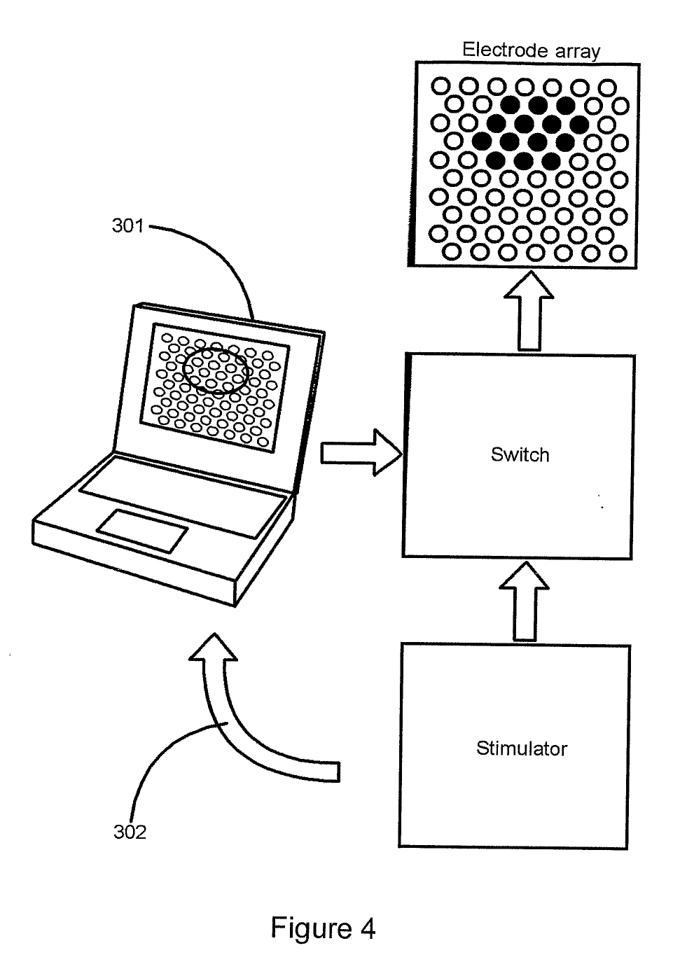

FIG. 4 illustrates a third embodiment of the invention, showing the operational interaction between its components, including a plan view of its array electrode; and

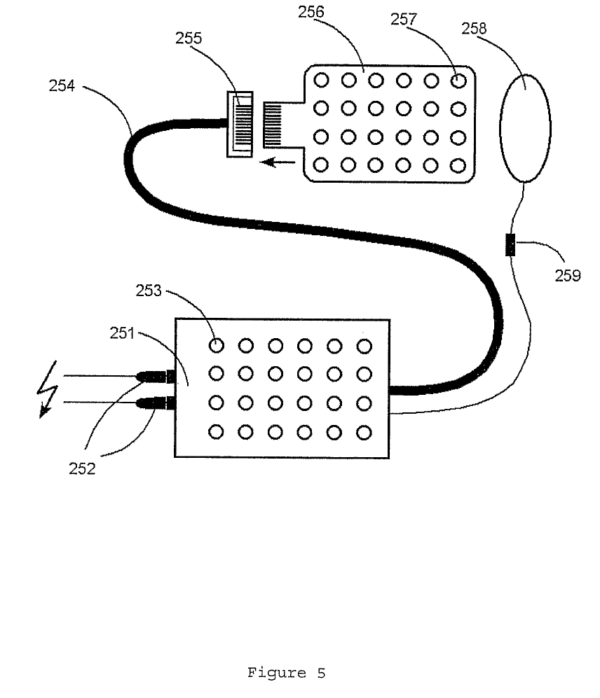

FIG. 5 shows a fourth embodiment according to the invention and shows an array electrode in plan view and a control unit also in plan view.

The apparatus of FIG. 1 consists of a controller 1 comprising an interface for the operator to control the apparatus, a switching means 2 which directs the output from a source of electrical stimulation signals or stimulator 3 to a surface array electrode 4 having multiple electrodes 5 forming an array and in galvanic contact with the skin. The array electrode is shown as rectangular, but may have any shape, e.g. be shaped to fit a specific body-part. The stimulator 3 can be any commercial or custom-made stimulator suitable for the particular application. The switch means 2 directs the output from the stimulator 3 to one or more of the multiple electrodes of the array electrode as determined by the controller 1. The controller allows sizing, shaping and locating of the active area 6 by the operator before or even during the operation.

The controller 1 allows selection of the most appropriate contact points for conducting the electrical signals from the stimulator 3 to the tissue. As illustrated below the interface represented by the controller 1 may be mechanical or electronic. Where it is electronic, it may comprise firmware and software to allow wireless communication of the controller 1 and a PC, Lap-top, or handheld computer for computerized setup.

Four embodiments of the invention are illustrated in FIGS. 2 to 5. One is an integrated user interface, controller and electrode shown in FIG. 2; in another the controller and user interface are separate from the electrode (FIG. 3); in that of FIG. 4, the controller and user interface is provided by means of a personal computer; and in the last, each electrode is controlled by a respective switch located on a remotely connected control box (FIG. 5).

A first embodiment is illustrated in FIG. 2. It consists of an array electrode having a substrate pad searing many electrodes 103. The substrate is adhesive on one face (front) on which are positioned the electrodes 103 and has miniature tactile switches 102 for manual operation embedded on the back of the array electrode, opposite the stimulating surface. Each switch connects or disconnects the corresponding electrode 103 in the array on the other side to/from a common electrical contact input 101. The switches 102 are designed so that the on/off state of each switch can be clearly seen by the user. In use the electrode is placed roughly over the right location on the skin, whereafter fine-tuning of the placement is done by activating the switches. The electrode is connected via contact 101 to a single channel of an electronic stimulator as would be any other stimulation electrode. The second electrode (counter electrode) in the circuit could be a single contact anode, or a similar array electrode.

After the electrode is adhered to the skin, by applying electrical stimulation to contact 101 and closing and opening switches 102, a practitioner or patient can find which electrodes in the array contribute most effectively to producing the desired stimulation effect, for instance to produce contraction of a muscle. These may lie adjacent one another or not. A selected set of electrodes may be of any shape and may include from one up to any number of the available electrodes and may form one or several contiguous groupings.

Although the illustrated array electrode has only one input connection 101 which is connectable in common with each of the electrodes 103 of the array, more than one such contact may be provided and then each may be connectable only to a particular respective group of the available electrodes 103. Such groups will usually be exclusive of one another (so that no electrode 103 is a member of more than one group) but if more complex switching and circuitry is employed may be overlapping (so that some but not all of the electrodes 103 fall into more than one group). Where groups overlap, preferably not all of the electrodes each fall into the same two groups.

In this and other embodiments, the electrode may be secured in place other than by adhesive, e.g. by a strap.

A second embodiment is illustrated in FIG. 3. A controller 201 is connected to a head-stage 202, by means of a thin and flexible cable 206. The head-stage includes a zero-insertion force multi-connector 203 which connects to an array electrode 204. The head-stage also includes an electronic switching circuit, and the number of conductors in the cable 206 can be kept at a minimum (3-4 leads) sufficient to communicate neuromuscular stimulating signals and data defining electrodes of the array to receive such signals. Each of the electrodes 205 is represented by a light emitting diode 207, or other indicator, on the controller 201. An active indicator light shows that the corresponding electrode contact is connected to the stimulator. An analog or digital joy-stick 212 controls the location, size and shape of the emulated electrode, indicated by the dashed lines 208. Whilst a user is pressing the “Size” button 211, the joystick controls the size and shape of the active area by moving one corner of the area while keeping the diagonal corner fixed e.g. in the upper right, as indicated in the figure. When the “Size” button 211 is released, the shape and size of the active area remains fixed and can then be moved around within the field by moving the joystick. A stimulator (not shown) is connected to the controller by means of a pair of leads 209 and connectors 210. The controller contains batteries and may be turned on and off by means of a button 213.

The array electrode 204 again has a substrate having one adhesive face carrying an array of the electrodes 205, which may be larger or smaller in terms of the number of electrodes than the array actually shown. Each electrode is provided with an electrical connection to a respective contact 220 of the zif connector plug portion, suitably by a conductive metal track formed on the substrate.

The circuitry in the head stage 202 is adapted to receive stimulation signals such as shaped biphasic pulses from the stimulator via the controller 201 and data signals from the controller 201 indicating to which electrodes selected from the whole array the signals should be connected. Based on these data signals, the circuitry of the head stage acts as a solid state switch means for each electrode of the array to connect the stimulation signals to the selected electrodes in common.

As in FIG. 2, the electrodes of the array electrode may form a single group from which a selection is made for connection to the same stimulation signals or may be divided into discrete or overlapping groups to receive the same or different stimulation signals.

Instead of the illustrated joystick arrangement, any alternative manual input method may be used for determining which electrodes in the array are to be connected. For instance, hardware switches (as in FIG. 2) may be provided on the controller 201 in an array mirroring the electrode array, as shown in more detail in FIG. 5.

The third embodiment includes a computer 301, which can be a standard PC, laptop or handheld. A touchpad, mouse, joystick, touch-sensitive screen or other standard pointing device serves as the input means for selecting the active area of the electrode. Different areas and shapes of the active area can be selected on the computer screen, which also shows the active area or areas during stimulation. The computer controls the switching box, either by a wired or a wireless connection (such as a standard infrared (IR) link or a radio link such as used by wireless keyboards and mice). Computer control adds the option of providing rapid changes in “electrode position”, which could be used for e.g. compensating for movement of the activated muscle. This is an often encountered problem when using standard surface electrodes. The motor point for a relaxed muscle may be different than that for a contracted muscle. The movement of the motor point can be entered into the computer, which can then compensate for this if it is informed of the timing and intensity of the stimulation. The latter is done by a trigger signal 302 being sent from the stimulator to the computer as shown or by feedback signal being sent from a movement sensor responding to changes in the muscle. The switch may be incorporated in a head stage connected to the array electrode as in FIG. 3.

Another embodiment is illustrated in FIG. 5. It consists of an array electrode 256 having a substrate bearing a plurality of electrodes 257.

The substrate is fabricated from a flexible material in order to be mounted on the selected part of the body. The substrate connects via a cable 254 to a controller box 251 with a zero insertion force multi-connector 255. The controller box comprises a plurality of tactile switches 253 that are positioned to match geometrically the positions of the plurality of electrodes. The tactile switches have respective indicators that by lighting show when each is in the on state. The controller box connects to the anode and cathode 252 of the stimulator that delivers monophasic, monophasic compensated, or biphasic bursts of stimulation pulses. The control box comprises a microcomputer that uses switches as inputs in order to connect the stimulator output to the selected electrodes in the array electrode.

All electrodes (cathodes) 257 stimulate with respect to a common anode 258 connected via a line connector 259, which anode may be a standard surface electrode or a similar array electrode.

In a modification of this embodiment is a device generally as shown in the FIG. 5 but where each switch in the array of mechanical switches shows its state by the position it is in. The controller in such a device does not require any power and can be made very cheaply.

All embodiments have been illustrated as for a single channel stimulator to be connected to the device. It is however simple to envision how the devices can be connected to a multi-channel stimulator. An extra button/dial on the user interface can allow the user to setup different electrode contact areas for different channels and the controller must then be able to differentiate between stimulation pulses entering on different input channels and direct them to the desired electrode contacts. The channels may be multiplexed, so that no two channels are active to send out current at precisely the same time.

An example of an application for such a multi-channel device could be a stimulation system for restoring hand function. To obtain even a rudimentary grasp function of a paralysed hand, it is necessary to apply four channels of stimulation (finger extension, finger flexion, thumb extension and thumb flexion). Some of (potentially all four) electrodes and the reference electrode in such a system can be replaced by a single large multi-channel electrode of the type described above.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’.

1. A nerve or muscle stimulation apparatus comprising:
a nerve stimulation array electrode comprising a substrate for application to the skin of a user bearing an array of electrodes arranged to be brought into electrical contact with the skin of a user when the substrate is positioned on said skin,
one or more input contacts, the or each said input contact being switchably electrically connected to a group of electrodes consisting of some or all of said electrodes, and
user operable switch means for performing said electrical connection by making and breaking electrical connection between the or each said input contact and any selected one or ones of its respective group of said electrodes.

2. Apparatus as claimed in claim 1, wherein said substrate bears an array of electrodes arranged in rows and columns.

3. Apparatus as claimed in claim 1, wherein there are at least 12 electrodes in the array.

4. Apparatus as claimed in claim 1, wherein there are at least 24 electrodes in the array.

5. Apparatus as claimed in claim 1, wherein the electrodes are evenly spread over the substrate.

6. Apparatus as claimed in claim 1, wherein the maximum space between adjacent electrodes is no more than 5 mm.

7. Apparatus as claimed in claim 6, wherein the maximum space between adjacent electrodes is no more than 2 mm.

8. Apparatus as claimed in claim 1, wherein the switch means is carried by the substrate.

9. Apparatus as claimed in claim 1, wherein the switch means comprises a respective manually operable switch associated with each electrode.

10. Apparatus as claimed in claim 1, wherein the switch means comprises:
an electronic user interface unit adapted to receive a user input representing an electrode selection and to communicate data signals representing said selection to an electronic switching unit; and
a said electronic switching unit adapted to receive said data signals and to perform connection of selected electrodes to said input contact(s).

11. Apparatus as claimed in claim 10, wherein said electronic user interface unit is adapted to accept programming to alter said selection of electrodes to accommodate said selection to alterations in the desired site of stimulation with time or user muscle movement.

12. Apparatus as claimed in claim 10, wherein said electronic switching unit is mounted to said substrate and wherein said electronic user interface unit is remote from said substrate and is wirelessly connected or in wired connection with said electronic switching unit.

13. Apparatus as claimed in claim 1, comprising in addition to said array electrode, one or more further electrodes connected to said switch means.

14. Apparatus as claimed in claim 1, wherein the user operable switch means is operable to make connection between the or a said input contact and a first electrode set constituted by a selected one or selected ones of its respective group of electrodes and with a second electrode set constituted by a selected one or selected ones of its respective group of electrodes, leaving disconnected at least one further electrode positioned between said first and second electrode sets.

15. Apparatus as claimed claim 1, further comprising a source of nerve or muscle stimulation signals connected to the or a said input contact.
